(19) United States
(12) Patent Application Publication
(10) Pub. No.: US 2025/0170247 A1
(43) Pub. Date: May 29, 2025

(54) CONJUGATES, AND USES THEREOF

(71) Applicant: CITY UNIVERSITY OF HONG KONG, Kowloon (HK)

(72) Inventors: Chun Kit KWOK, Kowloon (HK); Kun ZHANG, Kowloon (HK)

(21) Appl. No.: 18/963,702

(22) Filed: Nov. 28, 2024

Related U.S. Application Data
(60) Provisional application No. 63/604,171, filed on Nov. 29, 2023.

(51) Int. Cl. A61K 47/54; A61P 35/00

(52) U.S. Cl. CPC A61K 47/549; A61P 35/00

(57) ABSTRACT

Disclosed herein is a conjugate comprising a targeting moiety and a proteolytic moiety. According to some embodiments of the present disclosure, the targeting moiety comprises the nucleic acid of SEQ ID NO: 1, and a first conjugating group; and the proteolytic moiety comprises a ligand of E3 ubiquitin ligase, and a second conjugating group. The targeting moiety is linked to the proteolytic moiety via a click reaction occurred between the first and second conjugating groups. Also disclosed herein are methods of treating diseases by using the instant conjugate.

### CROSS-REFERENCES TO RELATED APPLICATIONS

This application relates to and claims the benefits of U.S. Provisional Application No. 63/604,171 filed Nov. 29, 2023; the content of the application is incorporated herein by reference in the entirety.

### SEQUENCE LISTING XML

The present application is being filed along with a Sequence Listing XML in electronic format. The Sequence Listing XML is provided as an XML file entitled HP0326US_SEQ, created Sep. 26, 2024, which is 5 Kb in size. The information in the electronic format of the Sequence Listing XML is incorporated herein by reference in its entirety.

### BACKGROUND OF THE INVENTION

### 1. Field of the Invention

The present disclosure in general relates to the field of pharmaceuticals. More particularly, the present disclosure relates to novel conjugates, and uses thereof in treating diseases, for example, Alzheimer's disease (AD) and cancer.

### 2. Description of Related Art

Ribonucleic acids (RNAs) fold into complex and dynamic structures to acquire amazing diversity and selectivity, allowing them to interact with proteins in cells to perform unique functions. RNA G-Quadruplex (rG4), assembled by multiple G-tetrads, is a crucial secondary structure of nucleic acids. Each G-quartet consists of four guanines arranged in a circular Hoogsteen hydrogen bond, and a central cation (K⁺>Na⁺>Li⁺) coordinates with the G-quartets stacking plane to further stabilize rG4 structure. A typical G-tetrad sequence consists of four consecutive guanine sequences separated by three runs of 1 to 7 nucleotides in length. In recent years, rG4 has become the focus of how the secondary structure of nucleic acids affects cellular processes. Studies have reported that rG4 plays an important role in genome regulation, including telomere maintenance, post-transcriptional regulation of messenger RNA (mRNA), and mature processing of non-coding RNA.

It is noteworthy that G4 binding protein (G4BP) is crucial for rG4-mediated gene regulation and has become one of the most promising therapeutic targets for drug development. For example, DEAH-Box helicase36 (DHX36) is known to bind and unfold rG4 in vitro. DHX36 is one of the G4-specific helicases, and the crystal structure indicates that it specifically binds to the parallel structure G4. DHX36 was reported as closely related to a variety of human diseases. Subcellular maps of human proteome show that DHX36 is overexpressed in a variety of cancer cells, including lung cancer and cervical cancer. In addition, it binds to the G4 containing long non-coding RNA FLJ39051, resulting in enhanced migration ability of colon cancer cells. Another partner of G4 structures, nucleolin (NCL), binds to lncRNA LUCATI rG4 to regulate the expression of MYC, thereby regulating and promoting the proliferation of colon cancer cells. According to previous reports, the rG4 motif is present in the 3′-untranslated region (3′UTR) of amyloid precursor protein (APP) transcript, and targeting the APP 3′UTR rG4 motif by L-RNA aptamer can control native APP protein expression. Recently, DHX36 was discovered to unfold rG4 in the 5′-untranslated region (5′UTR) and specifically regulates the translation of guanine nucleotide-binding protein G(i) subunit alpha-2 (Gnai2) mRNA, which is crucial for the regenerative ability of skeletal muscle stem cells (SCs). Therefore, the rG4-G4BP complex has become a drug target for interfering with rG4-mediated gene control in pathological states.

In view of the foregoing, there is a continuing interest in developing novel agents that specifically target and/or block the formation of the rG4-G4BP complex thereby treating rG4/G4BP-related diseases, e.g., Alzheimer's disease (AD) and cancers.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a conjugate comprising a targeting moiety and a proteolytic moiety. According to the embodiments of the present disclosure, the targeting moiety comprises a nucleic acid, and a first conjugating group linked to the nucleic acid, wherein the nucleic acid comprises the nucleotide sequence of “GGGUUGCGGAGGGUGGGCCU” (SEQ ID NO: 1); and the proteolytic moiety comprises a ligand of E3 ubiquitin ligase, and a second conjugating group linked to the ligand of E3 ubiquitin ligase. In these embodiments, the first and second conjugating groups are respectively selected from the group consisting of azide, alkyne, tetrazine, cyclooctene, and cyclooctyne groups; and the targeting moiety is linked to the proteolytic moiety via copper catalyzed azide-alkyne cycloaddition (CuAAC) reaction or strained-promoted azide-alkyne click chemistry (SPAAC) reaction, that occurred between the first conjugating group of the targeting moiety and the second conjugating group of the proteolytic moiety.

Optionally, the proteolytic moiety further comprises a linker linking the second conjugating group to the ligand of E3 ubiquitin ligase. Preferably, the linker comprises 1 to 5 repeats of ethylene glycol (EG) unit.

According to certain exemplary embodiments of the present disclosure, the ligand of E3 ubiquitin ligase is (2S,4R)-1-((S)-2-Amino-3,3-dimethylbutanoyl)-4-hydroxy-N-(4-(4-methylthiazol-5-yl)benzyl)pyrrolidine-2-carboxamide (AHPC), or pomalidomide. In some embodiments, the proteolytic moiety has the structure of formula (I) or formula (II),

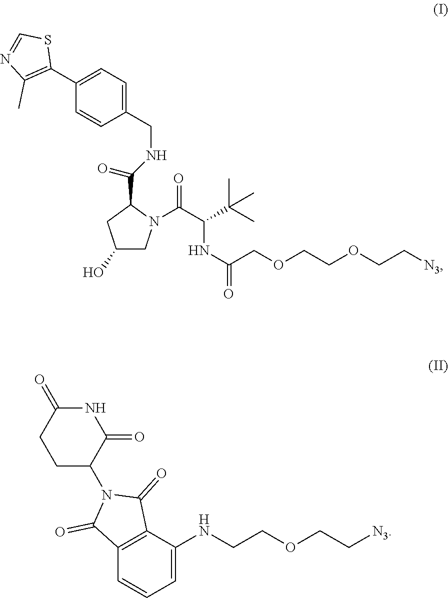 [SMILES: Cc1ncsc1-c1ccc(CNC(=O)[C@@H]2C[C@@H](O)CN2C(=O)[C@@H](NC(=O)COCCOCCN=[N+]=[N-])C(C)(C)C)cc1 ; [N-]=[N+]=NCCOCCNc1cccc2c1C(=O)N(C1CCC(=O)NC1=O)C2=O]

According to some exemplary embodiments, the first conjugating group is a hexynyl group, which is linked to the 5′ end of the nucleic acid.

In one embodiment, the present conjugate has the structure of formula (III),

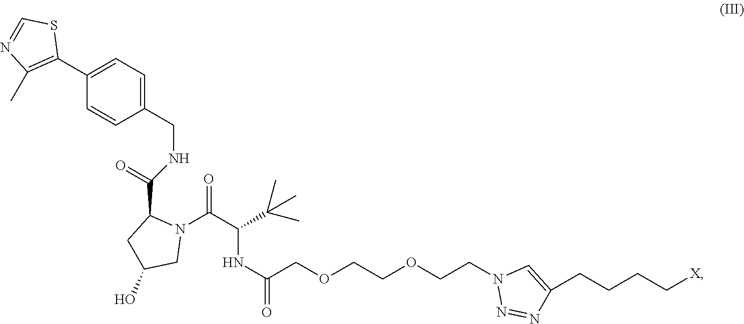

in which X is the nucleic acid.

In another embodiment the present conjugate has the structure of formula (IV),

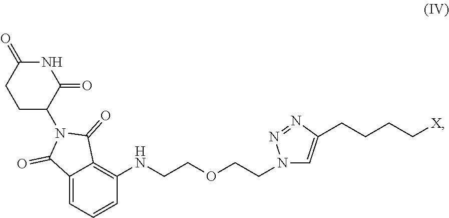

in which X is the nucleic acid.

Also disclosed herein is a method of treating a disease (especially, rG4/G4BP-related diseases) in a subject by using the present conjugate. According to some embodiments of the present disclosure, the method comprises administering to the subject an effective amount of the present conjugate to alleviate or ameliorate the symptoms associated with the disease. In one embodiment, the disease is AD. In another embodiment, the disease is a cancer.

The subject treatable with the present conjugate and/or method is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings.

FIG. 1A is a photograph of denaturing polyacrylamide gel electrophoresis (PAGE) that depicts the click efficiency of specified conjugate according to Example 1 of the present disclosure. 5Hexynyl_rG4: the rG4 oligonucleotide of SEQ ID NO: 1 (hTERC rG4 WT) modified with a 5′-Hexynyl. 5Hexynyl_rG4 mut: the rG4 oligonucleotide of SEQ ID NO: 2 (hTERC rG4 Mut) modified with a 5′-Hexynyl. rG4_A: a conjugate formed by linking 5′hexynyl-modified hTERC rG4 WT to azide-modified AHPC. rG4 mut_A: a conjugate formed by linking 5′hexynyl-modified hTERC rG4 Mut to azide-modified AHPC. rG4_P: a conjugate formed by linking 5′hexynyl-modified hTERC rG4 WT to azide-modified pomalidomide. rG4 mut_P: a conjugate formed by linking 5′hexynyl-modified hTERC rG4 Mut to azide-modified pomalidomide.

FIG. 1B is a histogram depicting the effect of specified conjugate on degrading DHX36 protein according to Example 1 of the present disclosure. * P<0.05, ** P<0.01. Normalized DHX36 expression was obtained from three biological replicates with the standard deviation as an error bar.

FIG. 2A is a line chart depicting the binding curve of FAM_rG4_A against RHAU53 peptide according to Example 2 of the present disclosure. The data were obtained from three biological replicates with the standard deviation as an error bar.

FIG. 2B is a line chart depicting the binding curve of FAM_dG4_A against RHAU53 peptide according to Example 2 of the present disclosure. The data were obtained from three biological replicates with the standard deviation as an error bar.

FIG. 2C is a line chart depicting the binding curve of FAM_rG4_A against DHX36 protein according to Example 2 of the present disclosure. The data were obtained from three biological replicates with the standard deviation as an error bar.

FIG. 2D is a line chart depicting the binding curve of FAM_rG4_A against DHX36 protein according to Example 2 of the present disclosure. The data were obtained from three biological replicates with the standard deviation as an error bar.

FIG. 3A is a histogram depicting the effect of rG4_A on degrading DHX36 protein according to Example 3 of the present disclosure. UTC: untreated control. ETC: empty transfection control. Normalized DHX36 expression was obtained from three biological replicates with the standard deviation as an error bar.

FIG. 3B is a photograph of western blot that depicts the effect of rG4_A or rG4 mut_A on DHX36 protein 0-48 hours post-transfection according to Example 3 of the present disclosure. 3-Actin: internal loading control.

FIG. 3C is a histogram depicting the effect of MG132 on blocking rG4_A induced DHX36 degradation according to Example 3 of the present disclosure. Normalized DHX36 expression was obtained from three biological replicates with the standard deviation as an error bar.

FIG. 3D is a histogram depicting the effect of rG4_A or rG4 mut_A on DHX36 degradation in specified cells according to Example 3 of the present disclosure. Normalized DHX36 expression was obtained from three biological replicates with the standard deviation as an error bar.

FIG. 4A is a schematic diagram depicting the design of dual luciferase reporter plasmid according to Example 4 of the present disclosure, in which APP rG4 WT sequence (SEQ ID NO: 3) or APP rG4 Mut sequence (SEQ ID NO: 4) was inserted into the 3′UTR of renilla luciferase.

FIGS. 4B and 4C are histograms respectively depicting the normalized luciferase activities of cells transfected with APP rG4 WT plasmid (FIG. 4B) or APP rG4 Mut plasmid (FIG. 4C) in the presence of rG4_A or rG4 mut_A according to Example 4 of the present disclosure. Normalized luciferase activities were obtained from three biological replicates with the standard deviation as an error bar. * P<0.05, ** P<0.01, NS: not significant.

FIGS. 4D and 4E are histograms respectively depicting the relative luciferase mRNA levels in cells transfected with APP rG4 WT plasmid (FIG. 4D) or APP rG4 Mut plasmid (FIG. 4E) in the presence of rG4_A or rG4 mut_A according to Example 4 of the present disclosure. Relative luciferase mRNA levels were obtained from three biological replicates with the standard deviation as an error bar. NS: not significant.

FIG. 5A is a schematic diagram depicting the design of APP native WT or APP native Mut plasmid according to Example 5 of the present disclosure, in which APP rG4 WT sequence (SEQ ID NO: 3) or APP rG4 Mut sequence (SEQ ID NO: 4) was inserted into the 3′UTR of the plasmid.

FIGS. 5B and 5C are photographs of western blot respectively depicting the expression of specified proteins in cells treated with 0, 25, 50 or 100 nM of rG4_A (FIG. 5B) or rG4 mut_A (FIG. 5C) according to Example 5 of the present disclosure. β-Actin: internal loading control. Mock: empty transfection control.

FIGS. 6A to 6C are photographs of western blot respectively depicting the effect of rG4_A or rG4 mut_A on the expression of DHX36 (FIGS. 6A and 6B) and Gnai2 (FIG. 6C) in the presence or absence of MG132 according to Example 6 of the present disclosure.

FIGS. 6D and 6E are histograms respectively depicting the effect of rG4_A or rG4 mut_A on the proliferation of C2C12 myoblasts (FIG. 6D) and satellite cells (SCs; FIG. 6D) according to Example 6 of the present disclosure. The relative EdU incorporation percentage was obtained from three biological replicates with the standard deviation as an error bar. * P<0.05, *** P<0.001.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “G-quadruplex structure” (G4 structure) as used herein refers to a four-stranded helical nucleic acid structure comprising multiple stacked G-tetrads, each of which consists of four guanine bases that associate in a cyclical manner through Hoogsteen hydrogen bonds and are further stabilized, through coordination to a cation in the center. The body of stacked G-tetrads, comprising a total of 2-8 layers, is collectively referred to as the G-tetrad core. Each of the four guanine columns constituting the G-tetrad core can arise from a single (continuous column), two, or four (discontinuous column) separate guanine stretch/stretches. The term “parallel G-quadruplex”, as used herein, relates to a G-quadruplex structure wherein all four strands point in the same direction.

The term “ligand” as used herein refers to a molecule that is recognized by and/or is capable of binding to a particular target protein (e.g., an E3 ubiquitin ligase). Depending on desired purpose, the ligand may be a natural ligand of the target protein or be a synthesized molecule having the ability to bind to or associate with the target protein. The ligand may bind to the target protein with any affinity, i.e., with high or low affinity, as long as it is capable of eliciting a desired activity or function of the target protein (e.g., a ubiquitin/proteasome-mediated degradation) once binding to the target protein.

As used herein, the term “targeting moiety” refers to the portion of a conjugate that binds to a target of interest (e.g., a G4BP of the present disclosure) thereby facilitates the transportation of the present conjugate to the interested target.

As used herein, the term “proteolytic moiety” refers to the portion of a conjugate that binds to E3 ubiquitin ligase, enabling the degradation of the target of interest (i.e., G4BP) via ubiquitin-proteasome system (UPS).

The term “link” refers to any means of connecting two components either via direct linkage or via indirect linkage between two components.

The terms “administered” and “administering” are used interchangeably herein to refer a mode of delivery, including, without limitation, intrathecal, intra-cerebellar, intratumoral, intravenous, intraarterial, intraperitoneal, or subcutaneous injection of an agent (e.g., the conjugate) of the present invention.

As used herein, the term “treat,” “treating” and “treatment” are interchangeable, and encompasses partially or completely preventing, ameliorating, mitigating and/or managing a symptom, a secondary disorder or a condition associated with a disease (e.g., a cancer or AD). The term “treating” as used herein refers to application or administration of the conjugate of the present disclosure to a subject, who has a symptom, a secondary disorder or a condition associated with the disease, with the purpose to partially or completely alleviate, ameliorate, relieve, delay onset of, inhibit progression of, reduce severity of, and/or reduce incidence of one or more symptoms, secondary disorders or features associated with the disease. Treatment may be administered to a subject who exhibits only early signs of such symptoms, disorder, and/or condition for the purpose of decreasing the risk of developing the symptoms, secondary disorders, and/or conditions associated with the disease. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives.

The term “subject” refers to a mammal including the human species that is treatable with the conjugate and/or method of the present invention. The term “subject” is intended to refer to both the male and female gender unless one gender is specifically indicated.

### II. Description of the Invention

The first aspect of the present disclosure is directed to a conjugate comprising a targeting moiety and a proteolytic moiety. According to some embodiments of the present disclosure, the targeting moiety comprises a nucleic acid, and a first conjugating group linked to the 5′ end or 3′ end of the nucleic acid. Regarding the proteolytic moiety, it comprises a ligand of E3 ubiquitin ligase, and a second conjugating group linked to the ligand of E3 ubiquitin ligase.

Preferably, the first and second conjugating groups are respectively selected from the group consisting of azide, alkyne, tetrazine, cyclooctene, and cyclooctyne groups. In this case, the targeting moiety is linked to the proteolytic moiety via a click reaction (i.e., CuAAC reaction, SPAAC reaction) occurred between the first conjugating group of the targeting moiety and the second conjugating group of the proteolytic moiety. According to one embodiment, one of the first and second conjugating groups is an alkyne group, and the other of the first and second conjugating groups is an azide group; in this embodiment, the targeting and proteolytic moieties are linked to each other via CuAAC reaction. According to another embodiment, one of the first and second conjugating groups is an azide group, and the other of the first and second conjugating groups is a cyclooctyne (e.g., a dibenzoazacyclooctyne (DBCO)) group; in this embodiment, the targeting and proteolytic moieties are linked to each other via SPAAC reaction.

According to the embodiments of the present disclosure, the nucleic acid comprises the nucleotide sequence of “GGGUUGCGGAGGGUGGGCCU” (SEQ ID NO: 1). In one exemplary embodiment, the first conjugating group is linked to the 5′ end of the nucleic acid.

According to some exemplary embodiments, the targeting moiety is in the form of an alkyne-modified nucleic acid, i.e., a nucleic acid (e.g., SEQ ID NO: 1) having a hexynyl group linked to its 5′ end; and the proteolytic moiety is in the form of an azide-modified E3 ligase ligand, i.e., an E3 ligase ligand having an azide group linked thereto. In these embodiments, the targeting moiety is linked to the proteolytic moiety via CuAAC reaction.

As could be appreciated, the ligand of E3 ligase may be any molecules exhibiting a binding affinity towards E3 ligase and transferring ubiquitin to targeted proteins. Non-limiting examples of the E3 ligase ligand suitable for use in the present invention include, lenalidomide, lenalidomide hemihydrate, pomalidomide, CC-885, eragidomide, thalidomide, thalidomide 4-fluoride, thalidomide 5-fluoride, PT-179, iberdomide, cemsidomide, golcadomide, AHPC (also known as “(S,R,S)-AHPC”), AHPC hydrochloride (also known as “(S,R,S)-AHPC hydrochloride”), and AHPC-Me hydrochloride (also known as “(S,R,S)-AHPC-Me hydrochloride”). In one embodiment, the ligand of E3 ligase is AHPC, which has the structure of

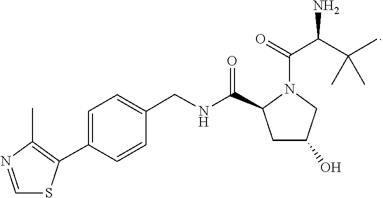 [SMILES: Cc1ncsc1-c1ccc(CNC(=O)[C@@H]2C[C@@H](O)CN2C(=O)[C@@H](N)CC(C)(C)C)cc1]

In another embodiment, the ligand of E3 ligase is pomalidomide, which has the structure of

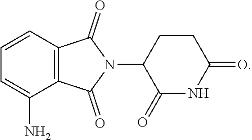 [SMILES: Nc1cccc2c1C(=O)N(C1CCC(=O)NC1=O)C2=O]

Optionally, the proteolytic moiety further comprises a linker (e.g., 1 to 5 repeat of PEG unit) linking the second conjugating group to the ligand of E3 ubiquitin ligase.

According to some embodiments of the present disclosure, the proteolytic moiety has the structure of formula (I),

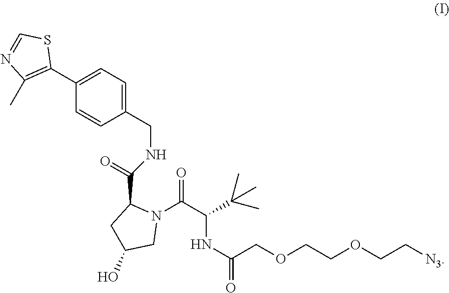 [SMILES: Cc1ncsc1-c1ccc(CNC(=O)[C@@H]2C[C@@H](O)CN2C(=O)[C@@H](NC(=O)COCCOCCN=[N+]=[N-])C(C)(C)C)cc1]

In these embodiments, the E3 ligase ligand is AHPC that is linked to an azide group via a linker comprising 2 repeats of EG unit.

According to one embodiment, the present conjugate has the structure of formula (III),

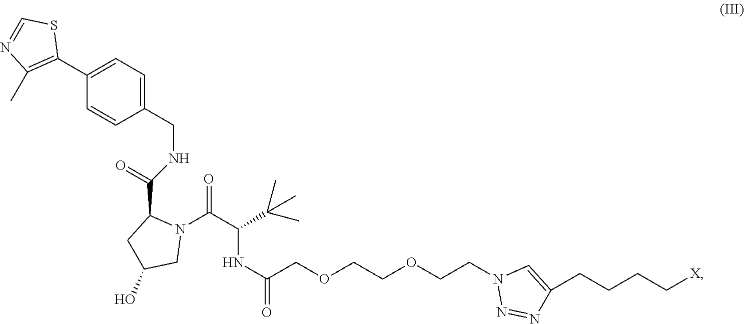

wherein X is the nucleic acid (e.g., SEQ ID NO. 1). In the embodiment, the proteolytic moiety having the structure of formula (I) is linked to a 5′-hexynyl modified nucleic acid via CuAAC reaction.

According to some embodiments of the present disclosure, the proteolytic moiety has the structure of formula (II),

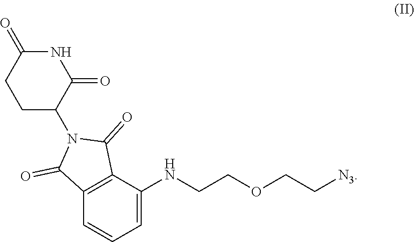 [SMILES: [N-]=[N+]=NCCOCCNc1cccc2c1C(=O)N(C1CCC(=O)NC1=O)C2=O]

In these embodiments the E3 ligase ligand is pomalidomide that is linked to an azide group via a linker comprising one EG unit.

According to one embodiment, the present conjugate has the structure of formula (IV),

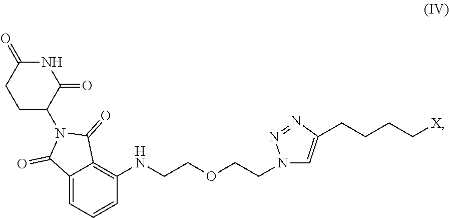

wherein X is the nucleic acid (e.g., SEQ ID NO: 1). In the embodiment, the proteolytic moiety having the structure of formula (II) is linked to a 5′-hexynyl modified nucleic acid via CuAAC reaction.

Preferably, the present conjugate is encapsulated by a lipid nanoparticle (LNP) that facilitates the transportation of the present conjugate into cells. As known in the art, LNP is a biocompatible vehicle of phospholipid monolayer structure, which wraps nucleic acid in lipid core and avoids degradation. The LNP usually have four components, including ionizable cationic phospholipid, neutral auxiliary phospholipid, cholesterol, and polyethylene glycol modified phospholipid. The methods for preparing LNPs are known in the art, for example, microfluidic preparation, or the use of microfluidic devices. Hence, the detailed description thereof is omitted herein for the sake of brevity. Alternatively, the present conjugate may be encapsulated/delivered by other delivery systems, for example, liposomes, lipid polycomplexes, polymer materials, micelles, polypeptides, protamine, or electroporation. See, for example, Mingyuan Li et al., European Journal of Medicinal Chemistry (2022), 227: 113910.

According to certain embodiments, upon the present conjugate entering the cells, the targeting moiety directly binds to G4BP, and the proteolytic moiety recruits the E3 ligase to facilitate the ubiquitination and subsequent degradation of the G4BP, thereby down-regulating or inhibiting the G4BP/rG4-medicated gene expression.

According to certain working examples, the present conjugate inhibits the expression of APP. According to some working examples, the present conjugate inhibits the expression of Gnai2.

The second aspect of the present disclosure is thus directed to a method of treating a disease (especially a disease associated with and/or caused by rG4 and/or G4BP overexpression) by using the present conjugate. The method comprises administering to a subject in need thereof an effective amount of the present conjugate to alleviate or ameliorate the symptoms associated with the disease. In some embodiments, the disease is cancer. In other embodiments, the disease is AD.

Examples of the cancer treatable with the present conjugate and/or method include, but are not limited to, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, head and neck squamous cell carcinoma, or a combination thereof.

As described above, the present conjugate is preferably encapsulated by a LNP. Alternatively, the present conjugate may be transported into cells via liposomes, lipid polycomplexes, polymer materials, micelles, polypeptides, protamine, and/or electroporation.

Depending on the intended purpose, the present conjugate may be administered to the subject via any suitable route, for example, transmucosal, intravenous, intraarterial, intramuscular, subcutaneous, intrathecal, intraperitoneal, or intra-cerebellar injection.

Basically, the subject treatable with the present method is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Click Reaction Between 5′-Hexynyl Oligonucleotides and Azide-Modified E3 Ligase Ligands

The following protocol was for alkyne-modified oligonucleotide and azide-containing compounds in 20 μL of dimethyl sulfoxide (DMSO) and nuclease-free water reaction mixture. Before the reaction, 1.6 μL of 5′-hexynyl oligonucleotides (from 1 mM stock in nuclease-free water) was denatured at 95° C. for 5 minutes and cooled for 10 minutes on ice. Triethylammonium acetate buffer (2 μL from 2 M stock in nuclease-free water, pH 7.0) was added to the denatured product to a final concentration of 0.2 M. Then, DMSO (2 μL) was added to the mixture followed by vortex. Azide compounds stock solution (6.4 μL from 1 mM stock in DMSO, final concentration: 320 M) was added to the mixture and vortex. 1 μL freshly prepared ascorbic acid (from 10 mM stock in nuclease-free water) and 1 μL Cu (II)-TBTA (from 10 mM stock in DMSO) were added to the mixture, and vortex briefly. An appropriate amount of DMSO and nuclease-free water (approximately 50:50 ratios in the final reaction mixture) was added to solubilize all the reagents used in the reaction mixture. The solution was degased by bubbling nitrogen gas for 30 seconds and tightly sealing it. The click reaction was kept on a shaker at 40° C. at 1,000 rpm for 6 hours. The rG4 conjugate was purified by spin column. The ethanol precipitation protocol was performed for the purification of dG4 conjugate (oligonucleotide nucleotide length<20 nt).

10% denaturing polyacrylamide gel was used to analyze click reaction efficiency after staining with SYBR® Gold, and yield determination was carried out by software. The synthesized conjugate was confirmed by matrix-assisted laser desorption/ionization time-of-flight (MALDI TOF/TOF) analyzer.

### Electrophoretic Mobility Shift Assay (EMSA)

All oligonucleotides were heated for 5 minutes at 75° C. and cooled down for 10 minutes on ice. Different concentrations of RHAU53 peptide (0-2 μM) and 5-carboxyfluorescein (FAM)-labeled oligonucleotides or G4-compound conjugate (30 nM) were mixed in the Tris-HCl buffer (25 mM Tris-HCl, pH 7.5, 150 mM KCl and 1 mM MgCl₂). The mixture was incubated at 37° C. for 30 minutes, and 5% glycerol was then added to each sample. Bound and unbound RNAs were separated on 10% native polyacrylamide gel in 0.5× Tris/Borate/EDTA (TBE) at 4° C., 150 V for 30 minutes. The gel was scanned by scanner at 500 V and quantified by software.

### Microscale Thermophoresis (MST)

Before the reaction, FAM labeled G4-compound conjugate was heated at 75° C. for 5 minutes and cooled down on the ice for 10 minutes. DHX36 protein was serially diluted to 16 samples from the highest concentration of 1 M. FAM-labeled oligonucleotides (40 nM) were added to each sample and incubated in Tris-HCl buffer (25 mM Tris-HCl, pH 7.5, 150 mM KCl, and 1 mM MgCl₂) at 37° C. for 30 minutes. Binding affinity was carried out on capillary tubes using an MST machine. The curve fitting and K_{d} determination were obtained from software.

Degradation Effect of rG4-Proteolytic Targeting Chimeras (rG4-PROTACs) on DHX36 by Western Blot Assay

1×10⁵ HeLa cells were seeded on a 12-well plate and cultured overnight. rG4-PROTACs or rG4 mut-PROTACs were transfected to cells for 24 hours using LIPOFECTAMINE® 2000 reagent. Cells were lysed in radioimmunoprecipitation assay (RIPA) buffer supplemented with a 1× protease inhibitor cocktail. Total protein was obtained by centrifuging at 13,000 rpm for 15 minutes at 4° C. 30 ng cell lysates of each sample were resolved by 8% sodium dodecyl sulfate polyacrylamide gel electrophoresis (SDS-PAGE) gel at 100V for 15 minutes and 120 V for 40 minutes. The protein blots were transferred to a polyvinylidene difluoride (PVDF) membrane at 100 V for 90 minutes at 4° C. and blocked with 5% non-fat milk for 1 hour at room temperature. Then, the membrane was incubated overnight with DHX36 primary antibody (1:1000) at 4° C. After washing 5 times with TBST buffer (Tris-buffered saline containing TWEEN® 20), the membrane was incubated with secondary antibody (1:1000) for 1 hour at room temperature and then washed 5 times with TBST buffer. The membrane was then detected by an imaging system and analyzed by software.

### Immunofluorescent Staining

2×10⁴ HeLa cells were seeded in a 35-mm diameter confocal dish and cultured overnight. FAM-labeled rG4-PROTACs (3 μmol) or FAM-labeled rG4 mut-PROTACs (3 μmol) were transfected to cells by LIPOFECTAMINE™ 2000 for 4 hours. The cells were fixed with 4% paraformaldehyde (PFA) for 15 minutes and washed 3 times with nuclease-free phosphate-buffered saline (PBS). Then, the cells were permeabilized with 1 mL 0.3% TRITON® X-100 at room temperature for 20 minutes. After washing 3 times with nuclease-free PBS, the cells were incubated with 1% bovine serum albumin (BSA) in PBS for 30 minutes to block unspecific binding of the antibodies. Then, the cells were incubated in the diluted DHX36 antibody (1:500) in 1% BSA overnight at 4° C. The cells were washed three times with PBS and incubated with ALEXA FLUOR™ 647 secondary antibody (1:500) in 1% BSA for 1 hour at room temperature in the dark. After washing 3 times with PBS, HeLa cells were stained with 4′,6-diamidino-2-phenylindole (DAPI) for 15 minutes. Cell imaging was performed with a confocal microscope.

### Dual-Luciferase Reporter Gene Assay

The DNA sequences encoding APP rG4 WT and APP rG4 Mut were respectively introduced into the 5′UTR of the Renilla luciferase gene in the psiCHECK-2 vector. 2×10⁴ HeLa cells were seeded in 96-well black-wall plates. 10 ng WT or Mut plasmids were co-transfected with rG4_A and rG4 mut_A (0, 50 nM, 100 nM, 200 nM) separately to cells by LIPOFECTAMINE™ 2000 and incubated for 48 hours. Luciferase activity was determined by a microplate reader. The Renilla activity was normalized to Firefly activity for data analysis.

### Quantitative Real-Time PCR (RT-PCR) Assay for Dual Luciferase Reporter Gene

Total RNA in 1×10⁵ HeLa cells was extracted by RNA extraction kits. 100 ng total RNA was reverse transcribed using a random primer. For PCR amplification procedures, the primers of reporter genes and cDNA were mixed with the SYBR® Green qPCR master mix. The RT-PCR was performed by a real-time PCR detection system.

Effect of rG4-PROTACs on Native APP Protein Expression

The DNA sequences encoding the APP full-length coding region, the Myc tag, and the APP rG4 WT or rG4 Mut were respectively introduced into the pEGFPN1 vector. 1×10⁵ HEK 293T cells were seeded in a 24-well plate and cultured overnight. 500 ng APP native WT or Mut plasmids were co-transfected with rG4_A and rG4 mut_A (0, 25 nM, 50 nM, 100 nM) separately to cells by LIPOFECTAMINE™ 2000 and incubated for 24 hours. DHX36, APP, and Myc protein expression were analyzed by western blot as the procedures described above. APP primary antibody (1:1000) and Myc primary antibody (1:1000) were used to incubate the PVDF membrane.

Downregulation of Gnai2 Translation by rG4-PROTACs

Mouse C2C12 myoblast cells (CRL-1772) were cultured in Dulbecco's Modified Eagle Medium (DMEM) supplemented with 10% fetal bovine serum (FBS) and 1% penicillin/streptomycin (complete media) at 37° C. in 5% CO₂. 5×10⁴ C2C12 cells were seeded in a 24-well plate and cultured overnight. rG4-PROTACs or rG4 mut-PROTACs (0, 50 nM, 100 nM, 150 nM, 200 nM) were transfected into cells for 24 hours using LIPOFECTAMINE™ 2000 reagent. DHX36 and Gnai2 protein expression were analyzed by western blot as the procedures described above. Gnai2 primary antibody (1:5000) was used in this assay.

EdU (5-ethynyl-2′-deoxyuridine) Cell Proliferation Assay

C2C12 myoblast cells and satellite cells were used in this assay. Satellite cells (SCs) were isolated from skeletal muscle tissues and cultured in Ham's F10 medium supplemented with 20% FBS and basic fibroblast growth factor (bFGF; 0.025 g/ml) (growth medium). EdU staining was followed by the manufacturer's protocols. In brief, 5×10⁴ C2C12 or SCs cells were seeded on coverslips and cultured overnight. rG4-PROTACs or rG4 mut-PROTACs (200 nM) were transfected into cells for 24 hours using LIPOFECTAMINE™ 2000 reagent. 10 mM EdU stock solution was diluted by complete medium to the concentration of 20 μM. The coverslips were transferred into the 6-well plate. Then, an equal volume of the 20 μM EdU solution was added to obtain a 10 M final solution and incubated for 60 minutes. The medium was removed followed by fixing the cells with 1 mL 3.7% PFA for 15 minutes at room temperature. The cells were washed 2 times with 3% BSA in PBS. 0.5% Triton® X-100 (1 mL) was added to the cells and then incubated for 20 minutes. The cells were washed 2 times with 3% BSA in PBS. Freshly prepared ALEXAFLUOR® 488 azide reaction cocktail was added to each well, and then the wells were incubated away from light for 30 minutes. The cells were washed 2 times with 3% BSA in PBS, and stained with DAPI for 15 minutes. Cell imaging was performed with a confocal microscope.

### Example 1 Design and Synthesis of rG4-PROTACs Targeting G4BP

It is known that human telomerase RNA component (hTERC) rG4 (5′-GGGUUGCGGAGGGUGGGCCU-3′; SEQ ID NO: 1) could strongly bind to RHAU-specific motif containing 53 amino acids (RHAU53) with nanomolar affinity. Accordingly, hTERC rG4 (hTERC rG4 WT; SEQ ID NO: 1) was selected as the warhead targeting G4BP; and the control sequence (hTERC rG4 Mut; GAAUUGCGGAGAAUGAACCU; SEQ ID NO: 2) was designed by mutating the key base G into A to break rG4 formation. Meanwhile, two widely validated E3 recruiting molecules, AHPC (a VHL ligand) and pomalidomide (a cereblon ligand), were used to respectively connect with the rG4 motif. Alkyne was attached to the 5′ end of rG4, and azide was introduced to derivatives of AHPC and pomalidomide. Four rG4-PROTACs (rG4_A, rG4 mut_A, rG4_P, and rG4 mut_P) were successfully synthesized using 5′hexynyl-modified RNA oligomers and azide-modified compounds via copper(I)-catalyzed click reaction (Table 1).

TABLE 1
rG4-PROTACs of the present disclosure

| Name | Structure | | Note |
|---|---|---|---|
| rG4_A | 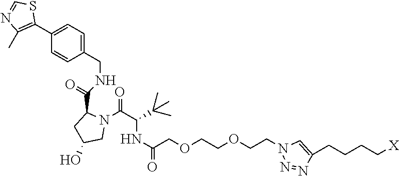 | Formula (III) | 5′hexynyl-modified hTERC rG4 WT was linked to azide-modified AHPC via click reaction |
| | wherein X is hTERC rG4 WT (SEQ ID NO: 1) | | |
| rG4 mut_A | 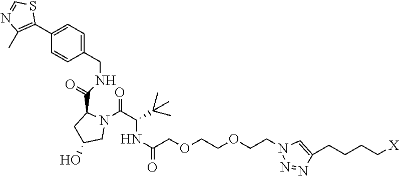 | Formula (III) | 5′hexynyl-modified hTERC rG4 Mut was linked to azide-modified AHPC via click reaction |
| | wherein X is hTERC rG4 Mut (SEQ ID NO: 2) | | |
| rG4_P | 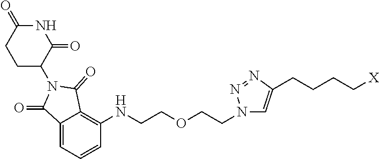 | Formula (IV) | 5′hexynyl-modified hTERC rG4 WT was linked to azide-modified pomalidomide via click reaction |
| | wherein X is hTERC rG4 WT (SEQ ID NO: 1) | | |
| rG4 mut_P | 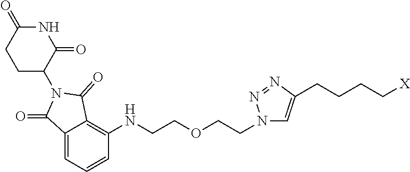 | Formula (IV) | 5′hexynyl-modified hTERC rG4 Mut was linked to azide-modified pomalidomide via click reaction |
| | wherein X is hTERC rG4 Mut (SEQ ID NO: 2) | | |

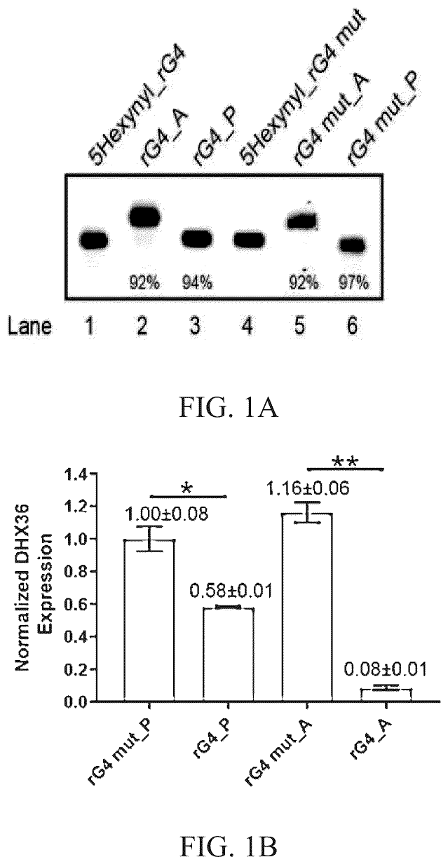

The click efficiency was monitored by denaturing PAGE and these conjugates achieved >90% of yields (FIG. 1A), which were confirmed by mass spectrometry (data not shown). Compared with rG4 mut ligand conjugates, treatment with rG4_A (50 nM, 24 h) resulted in 93% DHX36 degradation (FIG. 1B) in Hela cells, while rG4_P exhibited relatively weaker effects (42% degradation).

These results demonstrated that each of rG4-A and rG4-P was useful in degrading G4BP (DHX36). Since rG4-A worked more efficiently than rG4-P, it was chosen for the subsequent experiments.

### Example 2 Binding Affinity of rG4-PROTACs to RHAU53 and DHX36

To visualize the targeting effect of the rG4-PROTACs, the 3′ end of 5′Hexynyl_hTERC rG4 WT or Mut sequences (SEQ ID NO: 1 or 2) was labeled with fluorescein (FAM) and conjugated to the E3 ligase recruiter AHPC, hereafter referred to as FAM_rG4_A and FAM_rG4 mut_A. Recently, alkyne functionalized T95-2T dG4 and mutant sequences have been connected to E3 ligase-binding small molecules (dG4-PROTACs) to showcase potent degradation of RHAU by the proteasomal complex. To compare the binding affinity of rG4-PROTACs with dG4-PROTACs, FAM_dG4_A and FAM_dG4 mut_A conjugates were synthesized following the same click reaction method as rG4-PROTACs. These products were validated by mass spectrometry.

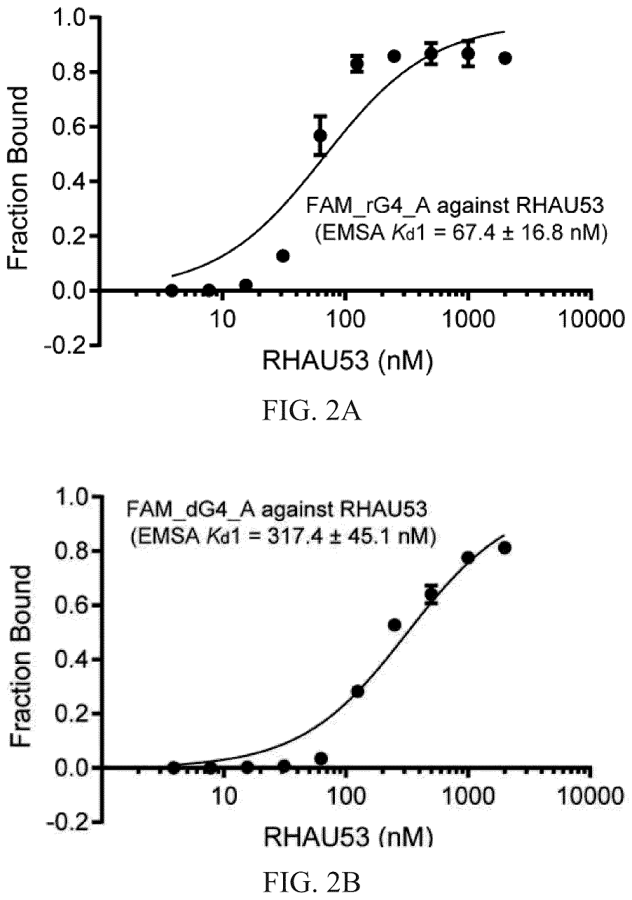

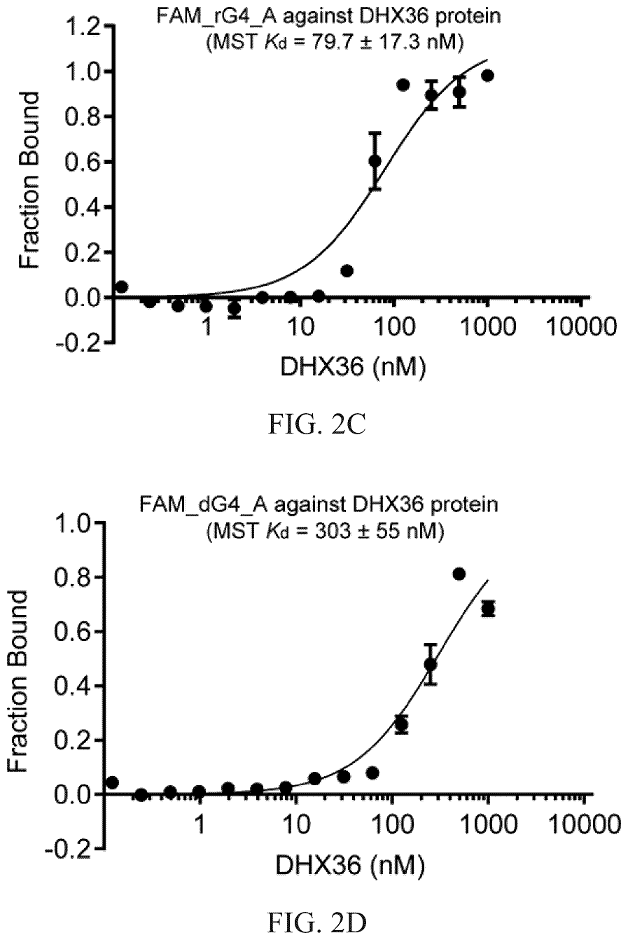

To evaluate the target recognition ability of the rG4-PROTACs, the binding assay of FAM_rG4_A to RHAU53 peptide was performed by EMSA. RHAU53 peptide, the truncated DHX36 fragment, is the most important core protein domain required for rG4-RHAU interaction. According to results of FIGS. 2A and 2B, the dissociation constant (K_{d}) of FAM_rG4_A was found to be 67.4±16.8 nM (FIG. 2A), which is approximately 4.7 times lower than that of A_dG4_FAM (317.4±45.1 nM; FIG. 2B).

Full-length DHX36 protein was then used to further confirm the recognition of the present rG4-PROTAC to G4BP via MST binding assay. According to the result of FIG. 2C, the K_{d} (79.7±17.3 nM) of FAM_rG4_A determined by MST binding assay was consistent with the EMSA result of FIG. 2A, demonstrating the strong interaction between FAM_rG4_A and DHX36. As described above, the affinity of A_dG4_FAM to RHAU53 was nearly 4.7 times higher (K_{d}=317.4±45.1 mM) than that of FAM_rG4_A (FIG. 2B). The interaction between A_dG4_FAM and DHX36 protein quantified by MST (K_{d}=303±55; FIG. 2D) confirmed the result, indicating a relatively weaker interaction with DHX36.

To investigate the binding specificity of G4-PROTACs, the EMSA assay was conducted using the non-G4 motif (FAM_rG4 mut_A and FAM_dG4 mut_A), and no binding was observed (data not shown), suggesting the targeting of G4-PROTACs to DHX36 is depending on rG4 formation.

Overall, rG4-PROTACs exhibited almost 3-fold improvement in binding affinity to DHX36 as compared to dG4-PROTACs, which could be effective tools for degrading rG4-binding proteins.

### Example 3 rG4-PROTACs Induced DHX36 Degradation in a Ubiquitination-Dependent Manner

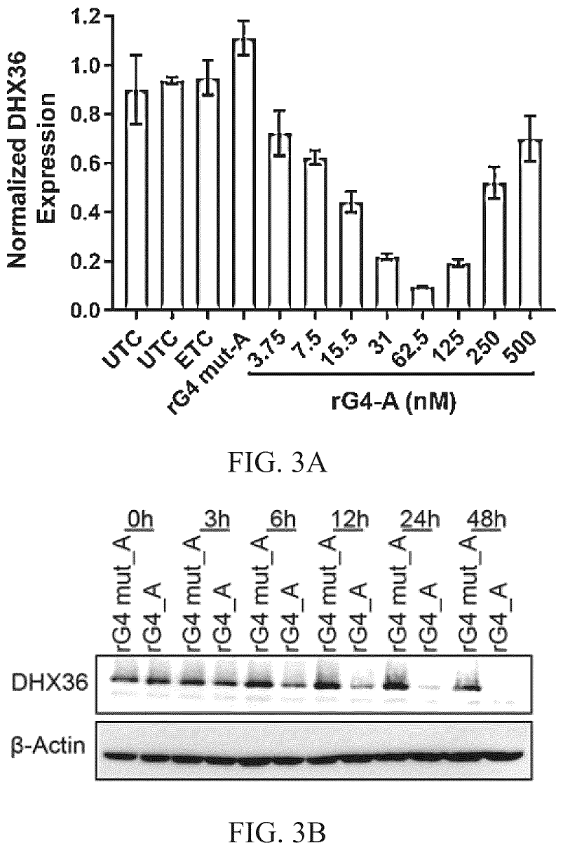

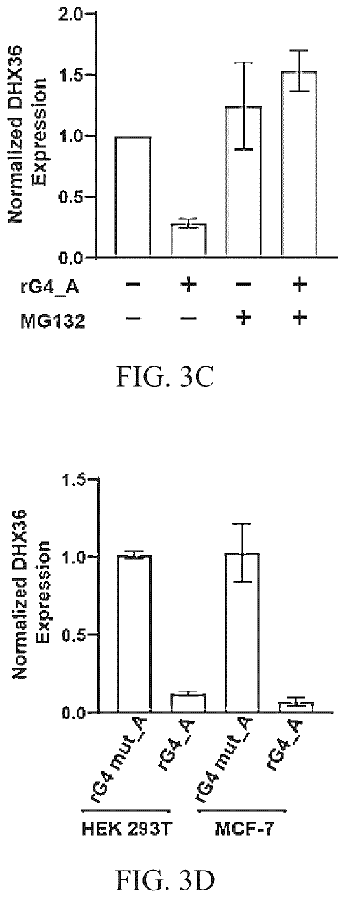

To verify whether rG4-PROTACs can enter cells, confocal microscopy was performed, and the results suggested that FAM-labeled rG4-PROTACs (FAM_rG4_A and FAM_rG4 mut_A) could be transfected successfully into Hela cell (data not shown). Meanwhile, cells treated with FAM_rG4_A exhibited strong fluorescent foci colocalized with endogenous DHX36, whereas little to no merged foci were observed in cells with FAM_rG4 mut_A transfection (data not shown), indicating that only the transfected rG4 motif degrader could target and interact with DHX36 protein. To characterize the degradation profile of rG4-PROTACs, varying concentrations of rG4_A were transfected into Hela cells. The western blot result indicated that the protein levels of DHX36 were significantly reduced by increasing concentrations of rG4_A, the highest DHX36 degradation (over 90%) was observed at 62.5 nM (FIG. 3A). Above 62.5 nM, protein degradation was attenuated with the increased dose of rG4-A (FIG. 3A), which corroborated those of previously reported PROTACs. The weaker effects at higher concentrations beyond 62.5 nM could be attributed to the cellular hook effect where the DHX36-rG4_A/rG4_A_E3 ligase binary interactions efficiently compete with DHX36-rG4_A_E3 ligase ternary interactions. As negative controls, neither untreated control (UTC), empty transfection control (ETC), nor rG4 mutant variant (rG4 mut_A) had a significant effect on DXH36 protein levels (FIG. 3A). In addition, the time dependency of rG4-A on DHX36 indicated that DHX36 reduction exceeded 50% within 6 hours of cells treated with 50 nM rG4_A, and maximal degradation was achieved at 95% at 48 hours (FIG. 3B).

Furthermore, rG4_A-induced degradation of DHX36 was blocked by the proteasome inhibitor MG132 (1 M), indicating that rG4 PROTACs mediated DHX36 degradation in a proteosome-dependent manner (FIG. 3C). Similarly, transfection of rG4_A (concentration 50 nM) to HEK 293T and MCF-7 cell lines also led to approximate 70% proteasomal degradation of DHX36 protein at the 24-hour time point (FIG. 3D). Notably, with the increase of dG4_A concentration, DHX36 protein expression was not significantly inhibited (data not shown). These data, which are in good agreement with those obtained with EMSA results (FIG. 2), demonstrated that the targeting effect and degradation efficiency of dG4-PROTACs were relatively weaker than rG4-PROTAC.

The effect of rG4-PROTACs was further compared with traditional DHX36 gene-silencing siRNA. Compared with the control siRNA (siNC)-treated sample, no significant degradation of DHX36 was observed after 24 hours of siRNA treatment, and it was not until 48 hours post-treatment that 85% declined in DHX36 protein was observed (data not shown). Accordingly, rG4-PROTACs work for a much shorter time (>50% reduction was achieved at the time point 6 hours and nearly complete depletion of DHX36 was observed in 24 hours) (FIG. 3B) than the siRNA method, which is an effective G4BP degradation tool.

Together, these findings support the view that rG4 motifs could be harnessed to construct G4BP targeting depredators, inducing the ubiquitination of DHX36 via the ubiquitin-proteasome system (UPS).

### Example 4 Applications of rG4-PROTACs in Controlling APP Gene Expression

It is known that the rG4 motif exists in the 3′UTR of APP mRNA. DHX36 is a well-studied member of the DEAD/H-BOX helicase family and involved in a variety of biological processes by binding and unwinding the G4 structure. The EMSA result indicated that the binding affinity of APP rG4 to DHX36 is strong (data not shown). Whether DHX36 exhibits the helicase effect on APP 3′UTR rG4 and rG4-PROTACs may induce APP rG4 formation to inhibit its gene expression by degrading DHX36 was examined in this example. To investigate this point, a FAM labeled APP WT_unwind oligomer which contained the APP 3′UTR rG4 motif and polyA tail was synthesized to perform the DHX36 unwinding assay. According to the analytic results, the incubation of the APP WT_unwind and APP WT_Trap RNA constructs formed a stronger duplex band and a lighter rG4 band after thermal denature in the absence of DHX36 (serving as a positive control in the assay; data not shown). Notably, upon DHX36 binding and unfolding APP rG4 in the presence of ATP, the APP WT_Trap RNA sequence was complementary pairing with the APP WT_unwind construct thus the duplex and rG4 bands were formed with similar intensity to the positive control (data not shown). The duplex cannot be formed in the absence of either DHX36 or ATP, or replacing ATP with AMP-PNP, a non-hydrolyzable analog of ATP (data not shown). Overall, the data demonstrated that DHX36 could bind and unfold APP 3′UTR rG4 in vitro.

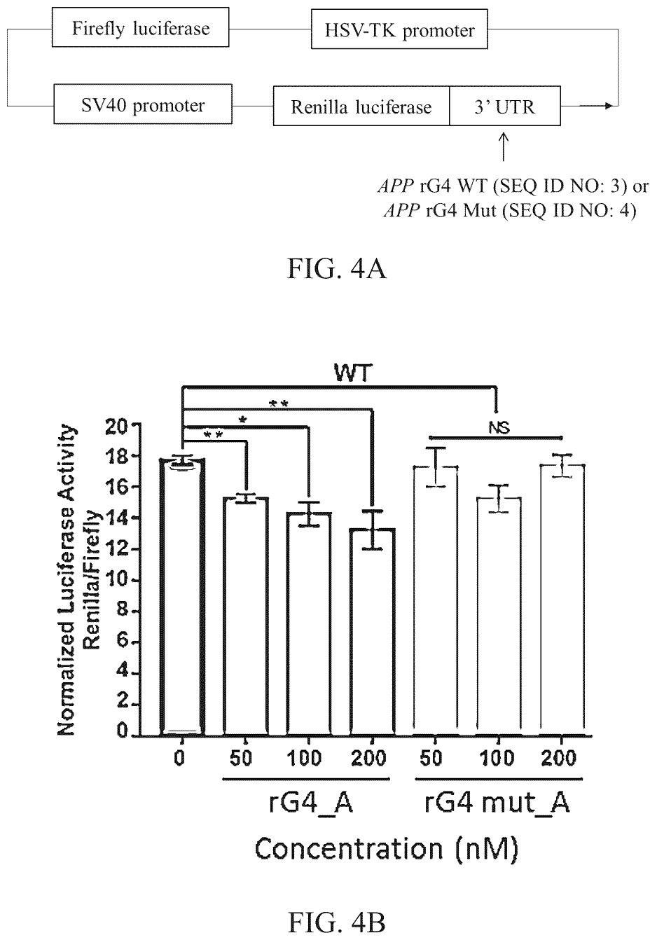

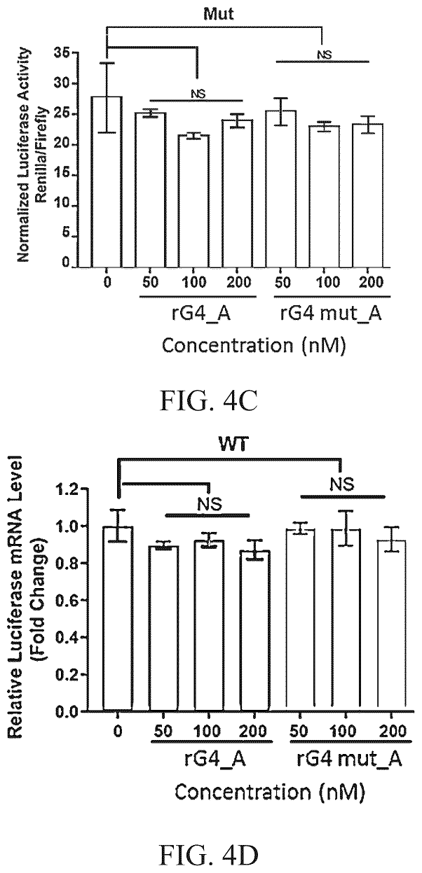

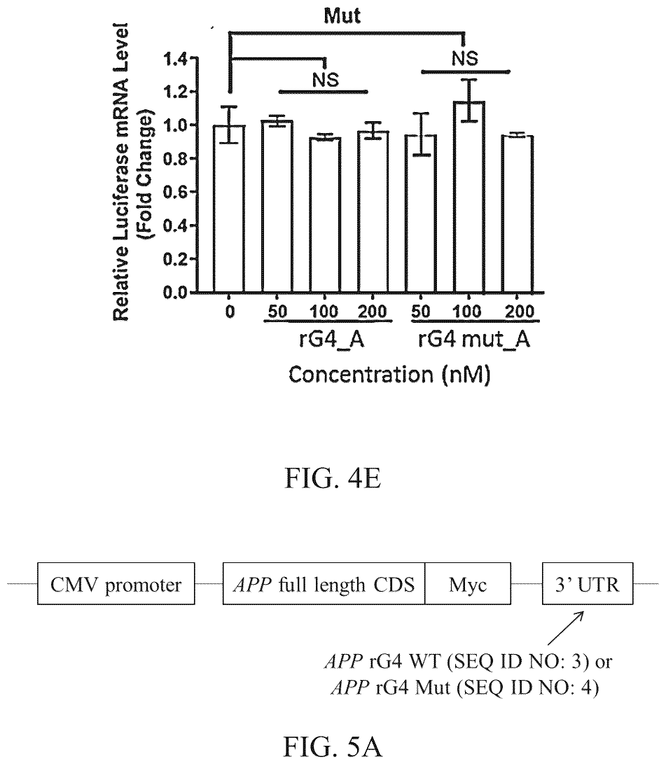

To investigate the effect of rG4-PROTACs on APP gene regulation in cells, a luciferase reporter plasmid was constructed by introducing APP rG4 WT motif (5′-CGGGGCGGGTGGGGAGGGGT-3′; SEQ ID NO: 3) or APP rG4 Mut motif (5′-CGAAGCGAGTGAAGAGAAGT-3′; SEQ ID NO: 4) into the 3′UTR of the Renilla gene, referred to as APP rG4 WT or APP rG4 Mut. The luciferase signal was normalized to the firefly luciferase in the same plasmid (FIG. 4A). The APP rG4 WT construct group showed a 36.03±1.02% reduction in normalized luciferase activity than that of the APP rG4 Mut construct (FIGS. 4B and 4C; the first columns), suggesting that the APP rG4 motif negatively regulates gene expression. Importantly, rG4_A inhibited the luciferase activity of APP rG4 WT construct (FIGS. 4B and 4C; columns 1-4), but not that of APP rG4 Mut construct (FIGS. 4B and 4C; columns 5-7), in a concentration-dependent manner. As the negative control, rG4_A can modulate luciferase activity neither in APP rG4 WT nor APP rG4 Mut constructs (FIGS. 4B and 4C). Finally, the mRNA level of dual luciferase genes was determined by RT-qPCR assay, and the data indicated no significant changes (FIGS. 4D and 4E), suggesting that the rG4_A affects the translational level but not the transcriptional level of target protein.

### Example 5 rG4-PROTACs Inhibited APP Native Protein Expression

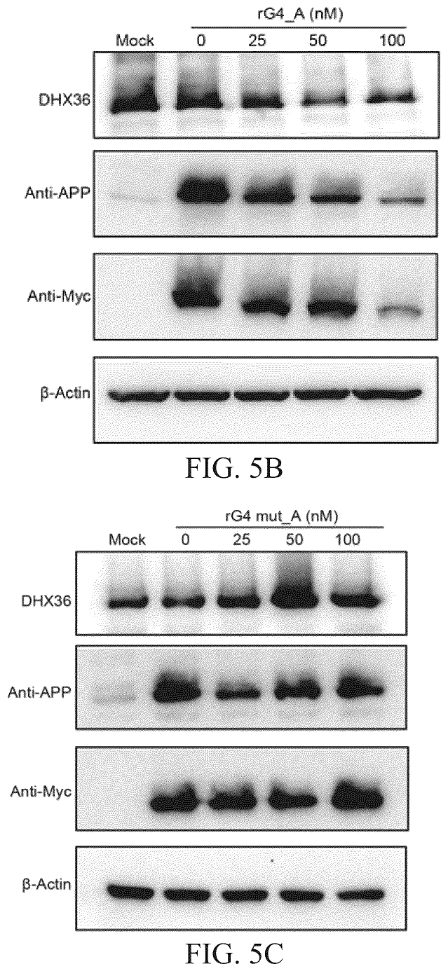

To further substantiate the effect of the rG4-PROTACs on rG4-mediated gene regulation above, the APP native protein expression was determined by constructs containing the APP full-length coding sequence (CDS), the Myc tag, and the rG4 wildtype or mutant motifs in the 3′UTR (FIG. 5A), coined to APP native WT and APP native Mut. The APP native WT plasmid was co-transfected with various concentrations of rG4_A or rG4 mut_A. The data of western blotting analysis revealed that the amount of native APP protein expression declined and coincided with an increased dose of rG4_A (FIG. 5B). By contrast, the normalized APP expression in APP native WT groups didn't exhibit a similar protein-suppressing effect in the presence of rG4 mut_A (FIG. 5C). The effect of rG4_A on gene expression in native APP Mut construct was also examined, and no difference was observed under rG4_A treatment (data not shown), which verified that the rG4-PROTACs only interfere with rG4-mediated gene regulation.

Taken together, these data suggested that rG4_A could induce the formation of rG4 motif by decreasing DHX36 protein, thereby negatively regulating rG4-mediated APP gene translation, resulting in the reduction of APP protein.

### Example 6 rG4-PROTACs Reduced Gani2 Protein Expression and Impacted Proliferative Capacity in Myoblast and Muscle Stem Cells

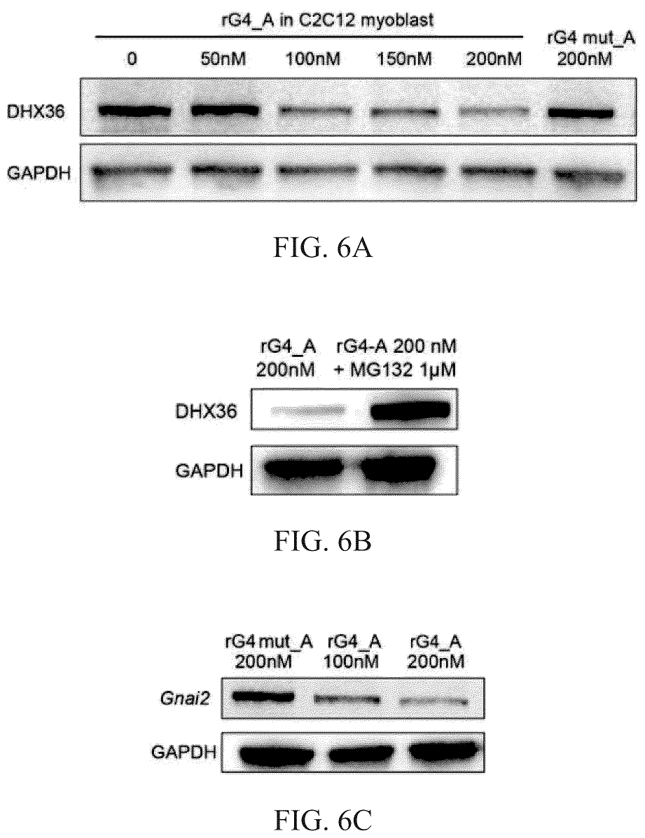

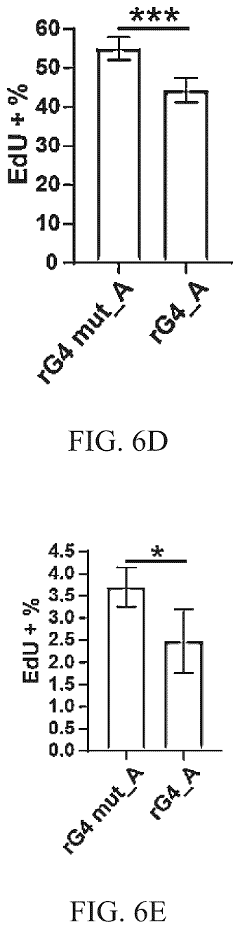

It is known that DHX36 specifically regulates the translation of Gnai2 mRNA by unwinding the rG4 motif at the 5′UTR, which is essential for the regenerative ability of SCs. In this example, the strong binding affinity of Gnai2 rG4 toward DHX36 protein was verified (data not shown), and the ability of rG4-PROTACs to modulate Gnai2 gene expression was tested by deletion of DHX36 protein cells. After increasing concentrations of rG4_A treatment, DHX36 showed a dose-dependent degradation while rG4 mut_A could not degrade DHX36 (FIG. 6A). Moreover, the degradation effect of rG4_A on DHX36 was blocked by the proteasome inhibitor MG132 (FIG. 6B). Concomitantly, the downstream effector gene Gnai2 also exhibited a concentration-dependent down-regulation in C2C12 myoblast (FIG. 6C), indicating that rG4_A promoted the formation of rG4 at Gnai2 5′UTR by degrading DHX36 protein, and triggered the translation inhibition of Gnai2 protein.

To assess the rG4_A effect on proliferative capacity, the EdU incorporation in C2C12 myoblast cultured for 2 days was measured. While 55.06%±2.94 of C2C12 myoblast were EdU⁺ in rG4 mut_A treated group, only 44.33%±3.08 of C2C12 myoblast were EdU⁺ in rG4_A treated group (FIG. 6D). A more obvious ability of rG4_A to inhibit proliferation was substantiated in SCs, in which 3.70%±0.44 of SCs were EdU⁺ in rG4 mut_A treated group, and 2.47%±0.72% were EdU⁺ in rG4_A treated group (FIG. 6E). The EdU assay suggested that the proliferative ability of SCs and C2C12 was compromised by DHX36 loss after rG4_A treatment.

In conclusion, the present disclosure provides two exemplary rG4-based proteolytic targeting chimeras (rG4-PROTACs) respectively designated as “rG4_A” and “rG4_P”. According to the examples of the present disclosure, each of the present rG4-PROTACs is capable of efficiently degrading G4BP and regulating the translation of rG4-containing transcripts in cells. In particular, DHX36, an extensively studied rG4 unwinding helicase could be deleted by rG4-PROTAC (rG4_A), resulting in a significant reduction of APP and Gnai2 protein expression. Hence, the present rG4-PROAC provides a potential means to treat various diseases (e.g., cancers and/or AD) via targeting abnormal rG4-G4BP complex.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A conjugate comprising,
a targeting moiety comprising a nucleic acid, and a first conjugating group linked to the nucleic acid, wherein the nucleic acid comprises the nucleotide sequence of “GGGUUGCGGAGGGUGGGCCU” (SEQ ID NO: 1); and
a proteolytic moiety comprising a ligand of E3 ubiquitin ligase, and a second conjugating group linked to the ligand of E3 ubiquitin ligase;
wherein
the first and second conjugating groups are respectively selected from the group consisting of azide, alkyne, tetrazine, cyclooctene, and cyclooctyne groups; and
the targeting moiety is linked to the proteolytic moiety via copper catalyzed azide-alkyne cycloaddition (CuAAC) reaction or strained-promoted azide-alkyne click chemistry (SPAAC) reaction that occurred between the first conjugating group of the targeting moiety and the second conjugating group of the proteolytic moiety.

2. The conjugate of claim 1, wherein the first conjugating group is a hexynyl group and is linked to the 5′ end of the nucleic acid.

3. The conjugate of claim 1, wherein the proteolytic moiety further comprises a linker linking the second conjugating group to the ligand of E3 ubiquitin ligase.

4. The conjugate of claim 3, wherein the linker comprises 1 to 5 repeats of ethylene glycol (EG) unit.

5. The conjugate of claim 1, wherein the ligand of E3 ubiquitin ligase is (2S,4R)-1-((S)-2-Amino-3,3-dimethylbutanoyl)-4-hydroxy-N-(4-(4-methylthiazol-5-yl)benzyl)pyrrolidine-2-carboxamide (AHPC), or pomalidomide.

6. The conjugate of claim 5, wherein the proteolytic moiety has the structure of formula (I) or formula (II),

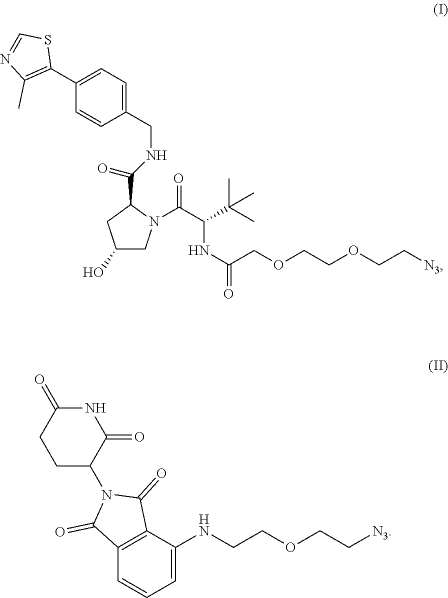 [SMILES: Cc1ncsc1-c1ccc(CNC(=O)[C@@H]2C[C@@H](O)CN2C(=O)[C@@H](NC(=O)COCCOCCN=[N+]=[N-])C(C)(C)C)cc1 ; [N-]=[N+]=NCCOCCNc1cccc2c1C(=O)N(C1CCC(=O)NC1=O)C2=O]

7. The conjugate of claim 6, wherein the conjugate has the structure of formula (III) or formula (IV), 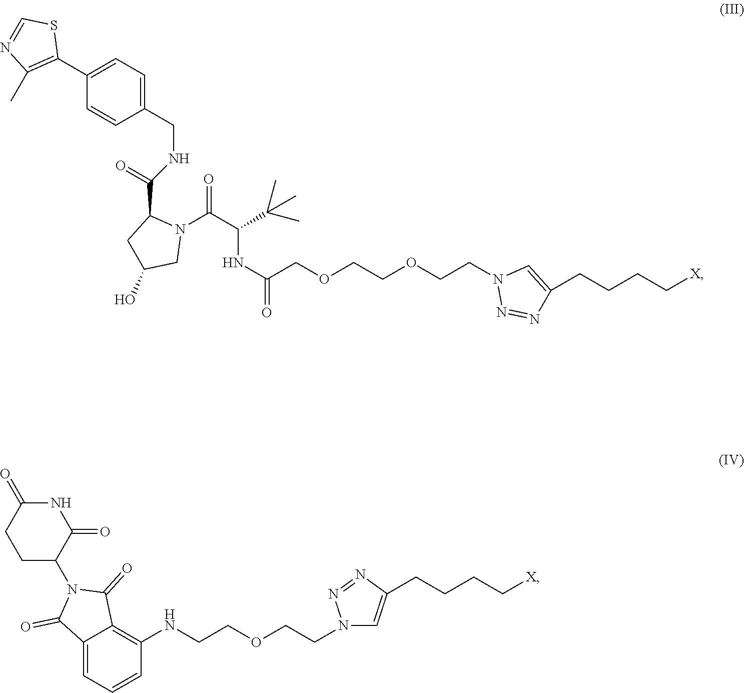
wherein X is the nucleic acid.

8. A method of treating Alzheimer's disease (AD) in a subject comprising administering to the subject an effective amount of the conjugate of claim 1 to alleviate or ameliorate the symptoms associated with the AD.

9. The method of claim 8, wherein the subject is a human.

10. A method of treating a cancer in a subject comprising administering to the subject an effective amount of the conjugate of claim 1 to alleviate or ameliorate the symptoms associated with the cancer.

11. The method of claim 10, wherein the subject is a human.


SEQUENCE LISTING (4 sequences)

SEQ ID NO: 1 - RNA, 20 nt, synthetic construct
  source 1..20: mol_type = other RNA
gggttgcggagggtgggcct

SEQ ID NO: 2 - RNA, 20 nt, synthetic construct
  source 1..20: mol_type = other RNA
gaattgcggagaatgaacct

SEQ ID NO: 3 - DNA, 20 bp, synthetic construct
  source 1..20: mol_type = other DNA
cggggcgggtggggaggggt

SEQ ID NO: 4 - DNA, 20 bp, synthetic construct
  source 1..20: mol_type = other DNA
cgaagcgagtgaagagaagt